(19) United States
(12) Patent Application Publication
(10) Pub. No.: US 2015/0051430 A1
(43) Pub. Date: Feb. 19, 2015

(54) Process and Apparatus for the Production of Paraxylene

(71) Applicant: ExxonMobil Chemical Patents Inc., Baytown, TX (US)

(72) Inventors: John Di-Yi Ou, Houston, TX (US); Dana L. Pilliod, League City, TX (US)

(21) Appl. No.: 14/335,009

(22) Filed: Jul. 18, 2014

Related U.S. Application Data
(60) Provisional application No. 61/866,288, filed on Aug. 15, 2013.

(51) Int. Cl. C07C 5/27

(52) U.S. Cl. CPC C07C 5/2729
USPC 585/302; 422/187

(57) ABSTRACT

A process for the production of paraxylene is disclosed, including utilizing a crystallization unit and a selective adsorption unit to produce paraxylene-rich streams comprising 99.7+wt % paraxylene and paraxylene-depleted streams comprising 10 to 15 wt % paraxylene. A portion of the paraxylene-depleted stream from the crystallization unit is passed through a liquid phase isomerization to produce an isomerized product containing xylenes at equilibrium or near-equilibrium concentration of 24 wt %.

### CROSS REFERENCE TO RELATED APPLICATIONS

This application claims the priority of U.S. Provisional Application No. 61/866,288, filed Aug. 15, 2013, the disclosure of which is incorporated herein by reference in its entirety.

### FIELD OF THE INVENTION

The invention relates to a process and apparatus for producing paraxylene including xylene isomerization.

### BACKGROUND OF THE INVENTION

The xylene isomers are important intermediates, which find wide and varied application in chemical syntheses. By way of example, paraxylene (PX) is a feedstock for terephthalic acid which finds use in the manufacture of synthetic fibers; metaxylene (MX) is used in the manufacture of dyes, and orthoxylene (OX) is used as a feedstock for phthalic anhydride, which finds use in the manufacture of plasticizers.

Xylenes are found in various fractions such as coal tar distillate, petroleum reformates and pyrolysis liquids in admixture with other compounds of like boiling points. The aromatic components are readily separated from non-aromatics by methods such as solvent extraction. A fraction consisting essentially of C8 aromatics may then be obtained readily such as by distillation. “C8 aromatics” means aromatic hydrocarbons having 8 carbon atoms, including particularly ethylbenzene and the xylene isomers paraxylene (p-xylene or PX), orthoxylene (o-xylene or OX), and metaxylene (m-xylene or MX).

While difficult to separate, due to their similar chemical structures, physical properties, and identical molecular weights, there are various methods used to separate the C8 isomers. For instance, orthoxylene is separable from other C8 aromatics by fractional distillation, and paraxylene is separable from other C8 aromatics by fractional crystallization or selective adsorption. Present demand is largely for paraxylene and it is desirable to convert metaxylene, the principal xylene present in the feed stream, and also orthoxylene, to paraxylene, to meet the market demand. At ordinary temperatures at which xylenes are processed in a typical petrochemical plant, the thermodynamic equilibrium content is approximately 24 mol % paraxylene, 56 mol % metaxylene, and 20 mol % orthoxylene, based on the total amount of xylenes in the feed.

Fractional crystallization is a method of separating components of a mixture and takes advantage of the differences between the freezing points and solubilities of the components at different temperatures. Due to its relatively higher freezing point, paraxylene can be separated as a solid from a C8 aromatic stream by fractional crystallization while the other components are recovered in a paraxylene-depleted filtrate stream. High paraxylene purity, a key property needed for satisfactory conversion of paraxylene to terephthalic acid and terephthalate esters, can be obtained by this type of fractional crystallization. U.S. Pat. No. 4,120,911 provides a description of this method. Commercially available fractional crystallization processes and apparatus include the crystallization isofining process, the continuous countercurrent crystallization process, direct CO₂ crystallizer, and scraped drum crystallizers. Due to high utility usage and the formation of a eutectic between paraxylene and metaxylene, it is usually more advantageous to use a feed with as high an initial paraxylene concentration as possible when using fractional crystallization to recover paraxylene.

An alternative xylene separation method uses molecular sieves, such as zeolites, to selectively adsorb paraxylene from a C8 aromatic feedstream to form a paraxylene-depleted effluent. The adsorbed paraxylene can then be desorbed by various ways such as heating, lowering the paraxylene partial pressure or stripping. (See generally U.S. Pat. Nos. 3,706,812, 3,732,325, and 4,886,929). Two commercially available processes, using molecular sieves to adsorb paraxylene are the PAREX™ and ELUXYL™ processes. In such molecular-sieve based adsorption processes, a higher amount of paraxylene, typically over 90%, compared with that from a fractional crystallization process, typically below 65%, may be recovered from the paraxylene present in a particular feed.

Paraxylene plants that employ both adsorption and crystallization units for paraxylene recovery often suffer from the problem of low energy efficiency and high material loss. This is mainly due to the crystallization unit's relatively low recovery of paraxylene, which produces a filtrate stream that contains a level of paraxylene in the range of 10 to 15 wt %. The liquid filtrate stream is subsequently processed in an energy-intensive vapor-phase isomerization unit to raise the paraxylene concentration to the equilibrium concentration of about 24 wt %. FIG. 1 shows a simplified schematic diagram for such a paraxylene plant.

In FIG. 1, the feed stream or streams 10 may come from a variety of sources, such as one or more sources selected from C8+ reformate, C8+ selective toluene disproportionation product, C8+ transalkylation product, C8+ toluene disproportionation product, and any other streams that contain C8 aromatics, such as products from benzene and/or toluene alkylation. These streams typically comprise the four C8 isomers and heavier aromatics (C9+ aromatics) which are processed along with a recycle stream 19 comprising isomerate, in one or more fractionation units 1 to remove C9+ aromatics. The C9+ and heavier aromatics could have an adverse effect on xylenes isomerization if not removed from the feed stream(s) by fractionation.

Continuing to refer to the prior art process shown in FIG. 1, the C8/C9+ aromatics fractionation unit 1 thus yields a C8 aromatics stream 41 which typically contains between 10 and 95 wt % paraxylene, and a bottom product 21 comprising C9+ aromatics, or optionally orthoxylene and C9+ aromatics, depending on the operation of the fractionation 1. The C8 aromatics stream 41 is split to selectively recover paraxylene by introducing a portion of the overhead 41 via conduit 11 to a selective adsorption unit 2 and a portion via conduit 12 to a crystallization unit 3. Paraxylene-enriched products 13 and 14, which may comprise as much as 99.7 wt % or even higher of paraxylene are recovered, with the balance of paraxylene-depleted C8 aromatics streams 15 and 16 passing to vapor phase xylenes isomerization unit 4. Vapor phase processes and catalysts therefore are per se well-known in the art.

Continuing with the system shown in FIG. 1, the xylenes isomerization product 17 passes to detoluenization fractionation unit 5 which removes C7 and lighter materials (C7−) overhead in stream 18 to yield C8 isomerate recycle stream 19, which is recycled to the C8/C9+ aromatics fractionation unit 1. Optionally, stream 21 of orthoxylene and C9+ aromatics can be sent to one or more fractionation unit 6 to produce an orthoxylene-rich stream 22 and a C9+ aromatics stream 23.

Another prior art process for paraxylene production that employs both selective adsorption and crystallization for paraxylene recovery is shown in FIG. 2. Feed stream 10 and isomerate recycle stream 24 are processed in C8 aromatics/C9+ aromatics fractionation unit 1 to remove C9+ aromatics or optionally orthoxylene and C9+ aromatics into stream 21, while feed stream 30 and recycle stream 25 are processed in a parallel C8 aromatics/C9+ aromatics fractionation unit 7 to yield C9+ aromatics stream 31. The C8 aromatics/C9+ aromatics fractionation units 1 and 7 thus yield C8 aromatics streams 12 and 11 which contain between 10 and 95 wt % paraxylene. In this schematic, stream 11 is processed to selectively recover paraxylene by a selective adsorption unit 2 and stream 12 is processed by a crystallization unit 3. For the selective adsorption unit 2, a paraxylene product of 99.7+wt % paraxylene is recovered as stream 13, with the balance of a paraxylene-depleted C8 aromatics raffinate stream 15 passing to vapor phase xylenes isomerization unit 4. For the crystallization unit 3, a paraxylene product of 99.7+wt % paraxylene is recovered as stream 14, with the balance of paraxylene-depleted C8 aromatics filtrate stream 16 passing to the same vapor phase xylenes isomerization unit 4.

Continuing to refer to the prior art process shown in FIG. 2, the isomerized product 17 passes to detoluenization fractionation unit 5 which removes C7 and lighter materials (C7−) in stream 18 to yield isomerate recycle stream 19. Isomerate recycle stream 19 is split into streams 24 and 25, which are recycled to the C8/C9+ aromatics fractionation units 1 and 7, respectively. Optionally, stream 21 of orthoxylene and C9+ aromatics can be sent to one or more fractionation unit 6 to produce an orthoxylene-rich stream 22 and a C9+ aromatics stream 23.

Known technologies integrate vapor phase xylene isomerization and liquid phase xylenes isomerization in paraxylene separation and isomerization loops. For example, U.S. Patent Publication No. 2012/0108868 describes separating paraxylene depleted (C8 aromatics) stream from paraxylene recovery through a parallel configuration of vapor phase xylene isomerization and liquid phase xylenes isomerization. U.S. Pat. No. 7,439,412 teaches using an isomerization unit under liquid phase conditions in order to recover one or more high purity xylene isomers, In an example, the product of the liquid phase isomerization unit is returned to the first fractionation tower in the system. See also U.S. Pat. No. 7,626,065. Similarly, U.S. Pat. No. 8,697,929 is directed to a xylenes isomerization process, including a liquid phase isomerization, for the production of equilibrium or near-equilibrium xylenes.

Due to the demand for paraxylene, there is an ongoing need for new processes and modifications to existing processes which significantly reduce energy consumption and prevents material loss.

### SUMMARY OF THE INVENTION

The invention is directed to a paraxylene production process and an apparatus for performing the process, in which a selective adsorption unit and a crystallization unit are operated in parallel to produce two separate paraxylene-enriched streams and two separate paraxylene-depleted streams. At least a portion of the paraxylene-depleted stream from the crystallization unit is sent to a liquid isomerization unit, rather than a vapor isomerization unit, to save energy and prevent material loss. The remainder of the paraxylene-depleted stream from the crystallization unit and the paraxylene-depleted stream from the selective adsorption unit is sent to vapor phase isomerization.

In embodiments, both the adsorption unit and the crystallization unit are fed from the overhead of the same fractionation unit. In other embodiments, the adsorption unit and the crystallization unit are fed from two separate fractionation units.

In embodiments, at least a portion of the liquid-phase isomerized product containing xylenes at equilibrium or near-equilibrium is then recycled to the crystallization unit. In other embodiments, at least a portion of the liquid-phase isomerized product containing xylenes at equilibrium or near-equilibrium is recycled to the initial fractionation.

It is an object of the invention to significantly reduce energy consumption by increasing the crystallization unit's recovery of paraxylene and utilizing liquid phase isomerization to isomerize at least a portion of the filtrate stream to produce an isomerized product containing xylenes at equilibrium or near-equilibrium.

These and other objects, features, embodiments and advantages will become apparent as reference is made to the following detailed description, preferred embodiments, examples, and appended claims.

### BRIEF DESCRIPTION OF THE DRAWINGS

In the accompanying drawings, like reference numerals are used to denote like parts.

FIGS. 1 and 2 are schematics illustrating prior art flow configurations for xylenes isomerization.

FIGS. 3 and 4 are schematics illustrating embodiments of the invention.

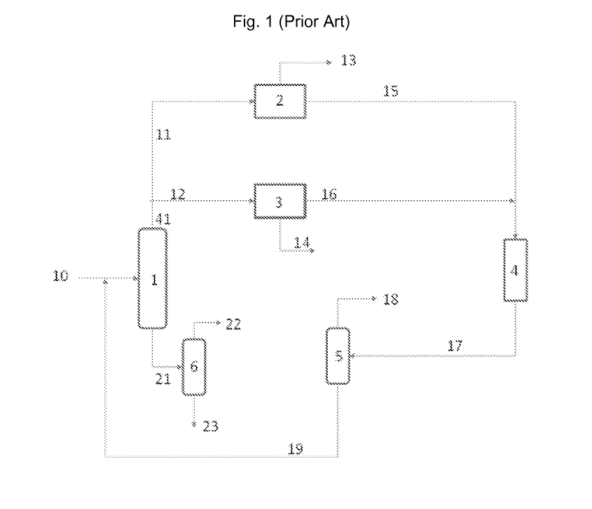

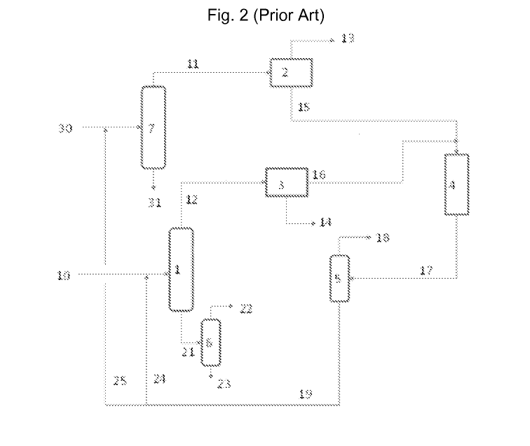

### DETAILED DESCRIPTION

According to the invention, an adsorption unit and a crystallization unit are operated in parallel to each produce a paraxylene-enriched stream and a paraxylene-depleted stream, followed by vapor phase isomerization of at least a portion of each of the paraxylene-depleted streams. The improvement includes introducing at least a portion of the paraxylene-depleted stream from the crystallization unit to a liquid phase isomerization unit to produce an isomerized product containing xylenes at equilibrium or near-equilibrium, which may be recycled directly to the crystallization unit. Sending at least a portion of the paraxylene-depleted filtrate from the crystallization unit to liquid phase isomerization, rather than vapor phase isomerization, saves energy and prevents material loss by avoiding the vaporization of the filtrate. Additionally, the liquid-phase isomerized product may be recycled to the crystallization unit rather than a fractionation unit, further saving energy and preventing material loss.

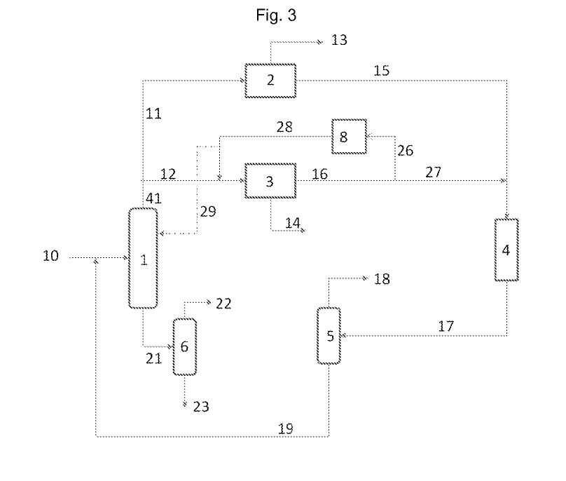

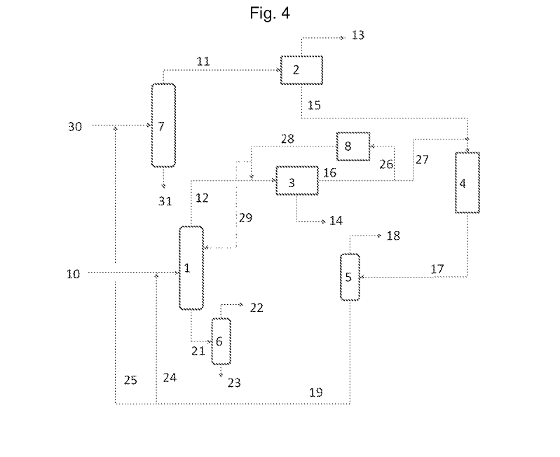

The invention may be better understood by reference to specific embodiments illustrated in FIG. 3 and FIG. 4. It will be understood by one of skill in the art in possession of the present disclosure that numerous modifications can be made and that the embodiment should not be taken as limiting to the invention described in the appended claims.

The feed stream(s) to the system shown in FIG. 3 and FIG. 4 may come from one or more sources comprising C8+ aromatic hydrocarbons, including C8+ reformate, C8+ selective toluene disproportionation product, C8+ transalkylation product, C8+ toluene disproportionation product, and any other streams that contain C8 aromatics such as products from toluene methylation with methanol.

In the flow configuration or schematic shown in FIG. 3, the feed stream or streams 10 is sent to one or more fractionation units 1 to remove C9+ aromatics, which yields a C8 aromatics stream 41 from the overhead which typically contains between 10 and 95 wt % paraxylene, and a bottom product 21 comprising C9+ aromatics and optionally orthoxylene. In a particular embodiment, a single fractionation unit 1 is used. The C8 aromatics stream 41 is split to selectively recover paraxylene by introducing a first C8 aromatics stream via conduit 11 to a selective adsorption unit 2 and a second C8 aromatics stream via conduit 12 to a crystallization unit 3. The selective adsorption unit 2 produces a first paraxylene-enriched stream 13 and a paraxylene-depleted raffinate stream 15. The crystallization unit 3 produces a second paraxylene-enriched stream 14 and a paraxylene-depleted filtrate stream 16. The new process configuration includes splitting the paraxylene-depleted filtrate stream 16 into streams 26 and 27. The paraxylene-depleted raffinate stream 15 and stream 27 are then passed to vapor phase xylenes isomerization unit 4 to produce a first isomerized product 17. The advantages of the present invention are achieved by isomerizing filtrate stream 26 in a liquid-phase isomerization unit 8 to produce a second isomerized product 28 containing xylenes at equilibrium or near-equilibrium concentration of 24 wt %, which is then recycled into the crystallizer unit 3. Therefore, instead of processing the liquid filtrate stream in an energy-intensive vapor-phase isomerization unit, at least a portion of the paraxylene-depleted filtrate 16 can be treated in a liquid-phase isomerization unit to reduce energy consumption and material loss. Optionally, a portion of the isomerized product 28 is recycled to fractionation unit 1 via stream 29. The first isomerized product 17 from the vapor phase isomerization unit 4 is then passed to detoluenization fractionation unit 5, which removes C7 and lighter materials (C7−) overhead in stream 18, to yield C8 isomerate recycle stream 19, which is recycled to the fractionation unit 1. Optionally, stream 21 of orthoxylene and C9+ aromatics can be sent to one or more fractionation units 6 to produce an orthoxylene-rich stream 22 and a C9+ aromatics stream 23.

In another embodiment shown in FIG. 4, the feed stream 30 is sent to at least one fractionation unit 7, preferably a first fractionation unit 7, from which a first C8 aromatics stream 11 is extracted overhead comprising the major portion of the metaxylene, paraxylene, ethylbenzene, and orthoxylene, and from which a stream 31 of C9+ hydrocarbons, and optionally orthoxylene, are extracted from the bottom. First C8 aromatics stream 11 is sent to a selective adsorption unit 2 to produce a first paraxylene-enriched stream 13 comprising 99.7+wt % paraxylene and a paraxylene-depleted raffinate stream 15, comprising 10 to 15 wt % paraxylene. Another feed stream 10 is sent to at least one fractionation unit 1, preferably a second fractionation unit 1, from which a second C8 aromatics stream 12 is extracted overhead comprising the major portion of the metaxylene, paraxylene, ethylbenzene, and orthoxylene, and from which a stream 21 of C9+ hydrocarbons, or optionally orthoxylene and C9+ hydrocarbons, is extracted from the bottom. Second C8 aromatics stream 12 is sent to at least one crystallization unit 3 to produce a second paraxylene-enriched stream 14 containing 99.7+wt % paraxylene, and a paraxylene-depleted filtrate stream 16 containing 10 to 15 wt % paraxylene. The paraxylene-depleted filtrate stream 16 is then split into streams 26 and 27. Paraxylene-depleted raffinate stream 15 and paraxylene-depleted filtrate stream 27 are sent to the vapor phase isomerization unit 4, producing a first isomerized product 17. Paraxylene-depleted filtrate stream 26 is isomerized in a liquid-phase isomerization unit 8 to produce a second isomerized product 28 containing xylenes at equilibrium and near-equilibrium concentration of 24 wt %. The second isomerized product 28 is then recycled to the crystallizer unit 3. Optionally, a portion of the isomerized product 28 is recycled to fractionation unit 1 via stream 29 or fractionation unit 7 (not shown). The first isomerized product 17 is sent to a detoluenization fractionation unit 5 to produce an overhead stream of hydrogen and C7− hydrocarbons 18 and a bottom stream 19 of isomerate containing C8+ hydrocarbons. Isomerate recycle stream 19 is recycled to the fractionation units 1 and 7 via streams 24 and 25. Optionally, stream 21 of orthoxylene and C9+ aromatics can be sent to one or more fractionation units 6 to produce an orthoxylene-rich stream 22 and a C9+ aromatics stream 23.

Regarding separation of xylenes in the paraxylene recovery, two preferred methods are fractional crystallization and selective adsorption, the details of which are per se known in the art. See U.S. Pat. No. 7,439,412, and references cited in the Background section. Likewise, the details of vapor phase xylenes isomerization and liquid phase xylenes isomerization are also per se known in the art. In this regard, see for example, U.S. Pat. No. 6,180,550.

An apparatus for the production of paraxylene according to the inventive process comprises at least one fractionation unit fluidly connected to a selective adsorption unit and a crystallization unit. In one preferred embodiment, one fractionation unit is connected to both the adsorption unit and crystallization unit such that the overhead from the fractionation unit is split, with one portion passing to the selective adsorption unit and another portion passing to the crystallization unit. In another preferred embodiment, a first fractionation unit is fluidly connected to provide an overhead to the adsorption unit and a second fractionation unit is fluidly connected to provide an overhead to the crystallization unit.

The adsorption unit and crystallization unit are fluidly connected to a vapor phase isomerization unit. The crystallization unit is also fluidly connected with a liquid phase isomerization unit to allow at least a portion of a paraxylene-depleted filtrate from the crystallization unit to pass to the liquid phase isomerization unit to produce a liquid phase isomerate recycle and the liquid phase isomerate recycle to pass back to the crystallization unit. The liquid phase isomerization unit is also in fluid communication with at least one of the fractionation units.

Non-limiting aspects and/or embodiments of the present disclosure:

### Embodiment 1

A process for producing paraxylene in which a first C8 aromatics stream is passed through a selective adsorption unit to produce a first paraxylene-enriched stream and a paraxylene-depleted raffinate stream and a second C8 aromatics stream is passed through a crystallization unit to produce a second paraxylene-enriched stream and a paraxylene-depleted filtrate stream wherein at least a portion of the paraxylene-depleted raffinate stream and at least a portion of the paraxylene-depleted filtrate stream is passed through a vapor phase isomerization unit to produce a first isomerized stream, the improvement comprising introducing a second portion of the paraxylene-depleted filtrate stream to a liquid phase isomerization unit to produce a second isomerized product containing xylenes at equilibrium or near-equilibrium.

### Embodiment 2

The process of Embodiment 1, wherein the first and second C8 aromatic streams are split from the overhead of a single fractionation unit.

### Embodiment 3

The process of Embodiment 1, wherein the first C8 aromatics stream is the overhead of a first fractionation unit and second C8 aromatics stream is the overhead of a second fractionation unit.

### Embodiment 4

The process of any one of Embodiments 1-3, wherein the second isomerized product is recycled to the crystallization unit.

### Embodiment 5

The process of Embodiment 2, wherein a portion of the second isomerized product is recycled to the single fractionation unit.

### Embodiment 6

The process of Embodiment 3, wherein a portion of the second isomerized product is recycled to at least one of the fractionation units.

### Embodiment 7

The process of any one of Embodiments 1-6, wherein the first isomerization product passes to a detoluenization fractionation unit to produce an isomerate recycle stream.

### Embodiment 8

The process of Embodiment 7, wherein at least a portion of the isomerate recycle stream is sent to a single fractionation unit to provide an overhead which split into the first and second C8 aromatics streams.

### Embodiment 9

The process of Embodiment 7, wherein a first portion of the isomerate recycle stream is sent to a first fractionation unit to provide overhead which is the first C8 aromatics stream, and a second portion of the isomerate recycle stream is sent to a second fractionation unit to provide an overhead which is the second C8 aromatics stream.

### Embodiment 10

A process for producing paraxylene comprising: (a) passing a first C8 aromatic stream to a selective adsorption unit to produce a paraxylene-enriched stream and a paraxylene-depleted raffinate stream; (b) isomerizing the paraxylene-depleted raffinate stream in a vapor phase isomerization unit; (c) passing a second C8 aromatic stream to a crystallization unit to produce a paraxylene-enriched stream and a paraxylene-depleted filtrate stream; (d) isomerizing a first portion of the paraxylene-depleted filtrate stream in a liquid phase isomerization unit to produce an isomerized product; (e) recycling the isomerized product to the crystallization unit; and (f) passing a second portion of the paraxylene-depleted filtrate stream to the vapor phase isomerization unit.

### Embodiment 11

The process of Embodiment 10, wherein the first and second C8 aromatics streams in steps (a) and (c) are split from the overhead of a single fractionation unit.

### Embodiment 12

The process of Embodiment 10 or Embodiment 11, wherein the first and second C8 aromatics streams in steps (a) and (c) are the overheads of two separate fractionation units.

### Embodiment 13

The process of Embodiment 11, wherein a portion of the isomerized product is recycled to the single fractionation unit.

### Embodiment 14

The process of Embodiment 12, wherein a portion of the isomerized product is recycled to at least one of the two separate fractionation units.

### Embodiment 15

An apparatus for the production of paraxylene comprising an adsorption unit and a crystallization unit fluidly connected to a vapor phase isomerization unit, wherein the crystallization unit produces a paraxylene-depleted filtrate, the improvement comprising a liquid phase isomerization unit fluidly connected with the crystallization unit, whereby at least a portion of the paraxylene-depleted filtrate is passed to in the liquid phase isomerization unit to produce a liquid phase isomerate recycle.

### Embodiment 16

The apparatus of Embodiment 15, wherein the liquid phase isomerization unit is fluidly connected to the crystallization unit so as to provide the liquid phase isomerate recycle to the crystallization unit.

### Embodiment 17

The apparatus of Embodiment 15 or Embodiment 16, including at least a first fractionation unit fluidly connected to provide an overhead to the adsorption unit and at least a second fractionation unit fluidly connected to provide an overhead to the crystallization unit, wherein the liquid phase isomerization unit is fluidly connected so as to provide the liquid phase isomerate recycle to at least one of the fractionation units.

### Embodiment 18

The apparatus of Embodiment 15 or Embodiment 16, including a fractionation unit fluidly connected to provide a portion of an overhead to the adsorption unit and the crystallization unit, wherein the liquid phase isomerization unit is fluidly connected so as to provide the liquid phase isomerate recycle to the fractionation unit.

While the present invention has been described and illustrated by reference to particular embodiments, those of ordinary skill in the art will appreciate that the invention lends itself to variations not necessary illustrated herein. All references cited herein are fully incorporated by reference to the extent such disclosure is not inconsistent with the invention and for all jurisdictions in which such incorporation is permitted.

What is claimed is:

1. A process for producing paraxylene in which a first C8 aromatics stream is passed through a selective adsorption unit to produce a first paraxylene-enriched stream and a paraxylene-depleted raffinate stream and a second C8 aromatics stream is passed through a crystallization unit to produce a second paraxylene-enriched stream and a paraxylene-depleted filtrate stream wherein at least a portion of the paraxylene-depleted raffinate stream and at least a portion of the paraxylene-depleted filtrate stream is passed through a vapor phase isomerization unit to produce a first isomerized stream, the improvement comprising introducing a second portion of the paraxylene-depleted filtrate stream to a liquid phase isomerization unit to produce a second isomerized product containing xylenes at equilibrium or near-equilibrium.

2. The process of claim 1, wherein the first and second C8 aromatic streams are split from the overhead of a single fractionation unit.

3. The process of claim 1, wherein the first C8 aromatics stream is the overhead of a first fractionation unit and second C8 aromatics stream is the overhead of a second fractionation unit.

4. The process of claim 1, wherein the second isomerized product is recycled to the crystallization unit.

5. The process of claim 2, wherein a portion of the second isomerized product is recycled to the single fractionation unit.

6. The process of claim 3, wherein a portion of the second isomerized product is recycled to at least one of the fractionation units.

7. The process of claim 1, wherein the first isomerization product passes to a detoluenization fractionation unit to produce an isomerate recycle stream.

8. The process of claim 7, wherein at least a portion of the isomerate recycle stream is sent to a single fractionation unit to provide an overhead which split into the first and second C8 aromatics streams.

9. The process of claim 7, wherein a first portion of the isomerate recycle stream is sent to a first fractionation unit to provide overhead which is the first C8 aromatics stream, and a second portion of the isomerate recycle stream is sent to a second fractionation unit to provide an overhead which is the second C8 aromatics stream.

10. A process for producing paraxylene comprising:
a) passing a first C8 aromatic stream to a selective adsorption unit to produce a paraxylene-enriched stream and a paraxylene-depleted raffinate stream,
b) isomerizing the paraxylene-depleted raffinate stream in a vapor phase isomerization unit,
c) passing a second C8 aromatic stream to a crystallization unit to produce a paraxylene-enriched stream and a paraxylene-depleted filtrate stream,
d) isomerizing a first portion of the paraxylene-depleted filtrate stream in a liquid phase isomerization unit to produce an isomerized product,
e) recycling the isomerized product to the crystallization unit, and
f) passing a second portion of the paraxylene-depleted filtrate stream to the vapor phase isomerization unit.

11. The process of claim 10, wherein the first and second C8 aromatics streams in steps (a) and (c) are split from the overhead of a single fractionation unit.

12. The process of claim 10, wherein the first and second C8 aromatics streams in steps (a) and (c) are the overheads of two separate fractionation units.

13. The process of claim 11, wherein a portion of the isomerized product is recycled to the single fractionation unit.

14. The process of claim 12, wherein a portion of the isomerized product is recycled to at least one of the two separate fractionation units.

15. An apparatus for the production of paraxylene comprising an adsorption unit and a crystallization unit fluidly connected to a vapor phase isomerization unit, wherein the crystallization unit produces a paraxylene-depleted filtrate, the improvement comprising a liquid phase isomerization unit fluidly connected with the crystallization unit, whereby at least a portion of the paraxylene-depleted filtrate is passed to in the liquid phase isomerization unit to produce a liquid phase isomerate recycle.

16. The apparatus of claim 15, wherein the liquid phase isomerization unit is fluidly connected to the crystallization unit so as to provide the liquid phase isomerate recycle to the crystallization unit.

17. The apparatus of claim 15, including at least a first fractionation unit fluidly connected to provide an overhead to the adsorption unit and at least a second fractionation unit fluidly connected to provide an overhead to the crystallization unit, wherein the liquid phase isomerization unit is fluidly connected so as to provide the liquid phase isomerate recycle to at least one of the fractionation units.

18. The apparatus of claim 15, including a fractionation unit fluidly connected to provide a portion of an overhead to the adsorption unit and the crystallization unit, wherein the liquid phase isomerization unit is fluidly connected so as to provide the liquid phase isomerate recycle to the fractionation unit.
